(19) United States
(12) Patent Application Publication
(10) Pub. No.: US 2023/0160881 A1
(43) Pub. Date: May 25, 2023

(54) HMGB1 RNA And Methods Therefor

(71) Applicant: NantOmics, LLC, Culver City, CA (US)

(72) Inventors: Patrick Soon-Shiong, Culver City, CA (US); Shahrooz Rabizadeh, Culver City, CA (US)

(21) Appl. No.: 16/646,734

(22) PCT Filed: Sep. 14, 2018
(86) PCT No.: PCT/US2018/051181

§ 371 Date: Mar. 12, 2020

Related U.S. Application Data
(60) Provisional application No. 62/559,234, filed on Sep. 15, 2017.

(51) Int. Cl. G01N 33/53; C12Q 1/6886; A61K 45/06

(52) U.S. Cl. CPC G01N 33/5308; C12Q 1/6886; A61K 45/06; C12Q 2600/118; C12Q 2600/158

(57) ABSTRACT

Methods for and uses of cell free RNA for determining prognosis of a cancer immunotherapy or for identifying a location of a tumor that is susceptible to a cancer immunotherapy are disclosed. A bodily fluid of a cancer patient treated with the cancer immunotherapy is obtained and cell free RNA is isolated from the bodily fluid. The amount of cell free RNA of at least one cancer related gene in the bodily fluid of the patient is identified, and the quantity of the cell free RNA is associated with the prognosis of the cancer immunotherapy. In some embodiments, the cell free RNA of at least one cancer related gene is cell-type specific or tumor-specific such that characterization of the cell free RNA identifies the location of the tumor.

This application claims priority to our co-pending US provisional application having the Ser. No. 62/559,234, filed Sep. 15, 2017, which is incorporated herein in its entirety.

### FIELD OF THE INVENTION

The field of the invention is cancer therapy, especially as it relates to immunotherapy with oncolytic viruses.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

All publications and patent applications herein are incorporated by reference to the same extent as if each individual publication or patent application were specifically and individually indicated to be incorporated by reference. Where a definition or use of a term in an incorporated reference is inconsistent or contrary to the definition of that term provided herein, the definition of that term provided herein applies and the definition of that term in the reference does not apply.

Prognosis of cancer in a patient treated with one or more cancer therapy can be determined by evaluating the effectiveness of the cancer therapy, which in turn, provides a helpful guidance to develop a future treatment plan. Traditional methods of determining prognosis of cancer includes direct/indirect measurement and examination of tumor size, depth of invasion, parametrical involvement and/or histology of the cancer tissue, which may not show significant changes in early phase of the cancer therapy and also often invasive. More recently, attention has been drawn to free circulating proteins in the serum as indicator of disease prognosis. For example, free circulating High molecular group box 1 (HMGB1, a highly conserved member of the HMG-box-family), has been found to be released from necrotic or damaged cells or from active immune cells, which can be used as a clinical biomarker for prediction and prognosis of malignant and autoimmune disease. For another example, the protein expression level extracellular domain of RAGE (receptor for advanced glycation end products), which can be a decoy receptor for HMGB1, was decreased in the serum of patients having acute autoimmune disease (e.g., Kawasaki disease, etc.).

However, most studies detecting free circulating molecules in the patient's serum are limited to detect protein expression level in the serum, which may not provide accurate information when the expression level of the free circulating protein is low or available detection tool (e.g., antibodies, etc.) is not sensitive. In addition, while a few studies attempting to detect mRNA level of several inflammation-related proteins in the patients' serum, those studies are limited to post-surgical procedure, which does not provide any link to the effectiveness of immune therapy in patients.

Thus, there remains a need for improved methods and compositions that correlate a readily available biomarker with the likely treatment outcome of cancer immune therapy.

### SUMMARY OF THE INVENTION

The inventive subject matter is directed to various compositions and methods of use of cell free RNA to determine prognosis of treatment outcome of cancer immunotherapy, to identify a location of a tumor that is susceptible to a cancer immunotherapy, to detect autophagy after cancer immune therapy, and to identify a compound effective to revert immune therapy resistant tumor cell to immune therapy sensitive tumor cell.

Thus, in one aspect of the inventive subject matter, the inventors contemplate a method for determining prognosis of cancer immunotherapy. In this method, a bodily fluid of a patient undergoing cancer immunotherapy is obtained. Most typically, the bodily fluid is selected from a group consisting of blood, serum, plasma, mucus, cerebrospinal fluid, and urine. In some embodiments, the bodily fluid of the patient is obtained at least 24 hours after the treatment with the cancer immunotherapy. From the patient bodily fluid, a quantity of a cell free RNA of at least one cancer related gene is identified. Typically, the step of identifying the quantity includes amplifying a signal of cell free RNA by real time, quantitative RT-PCR. In some embodiments, the quantity of the cell free RNA is an indicative of immune response activation in the patient.

Then, the quantity of the cell free RNA is associated with the prognosis of the cancer immunotherapy. In some embodiments, the step of associating comprises identification of an NK cell activation, identification of a T-cell mediated immune response activation, and identification of autophagy. The inventors also contemplate that this method can be used for identifying a molecular marker for determining prognosis of cancer immunotherapy.

Preferably, the cancer immunotherapy is a treatment of the individual with a recombinant neoepitope vaccine, a treatment of the individual with an oncolytic virus, and/or a treatment of the individual with a checkpoint inhibitor. Also preferably, the cell free RNA is at least one of ctRNA or cfRNA. In some embodiments, the cell free RNA is mRNA encoding an inflammation-related protein. In other embodiments, the cell free RNA is mRNA encoding a protein selected from a group consisting of HMGB1, MUC1, VWF, MMP, CRP, PBEF1 TNF-α, TGF-β, PDGFA, and hTERT. Where the cell free RNA is mRNA encoding HMGB1, the mRNA encoding HMGB1 comprises a plurality of alternative splicing variants, and/or is generated in a cancer cell.

In some embodiments, the cell free RNA is a regulatory non-coding RNA. In such embodiments, expression of the regulatory non-coding RNA may modulate expression of mRNA encoding a protein selected from a group consisting of HMGB1, MUC1, VWF, MMP, CRP, PBEF1 TNF-α, TGF-β, PDGFA, and hTERT.

In some embodiments, the method further includes steps of obtaining a bodily fluid of a healthy individual, identifying a quantity of the cell free RNA of the at least one cancer related gene in the bodily fluid of the healthy individual, and comparing the quantity of the cell free RNA in the bodily fluid of the healthy individual with the quantity of the cell free RNA in the bodily fluid of the patient. In other embodiments, the method further includes steps of obtaining a bodily fluid of a patient before treating the patient with the cancer immunotherapy, identifying a pre-treatment quantity of the cell free RNA of the at least one cancer related gene in the bodily fluid of the patient, and comparing the pre-treatment quantity with the quantity of the cell free RNA in the bodily fluid of the patient.

Another aspect of the inventive subject matter includes a use of a cell free RNA encoding at least one cancer related gene in a bodily fluid of a patient for determining prognosis of a cancer immunotherapy according to the method described above.

In still another aspect of the inventive subject matter, the inventors contemplate a method for identifying a location of a tumor that is susceptible to a cancer immunotherapy. In this method, a bodily fluid of a patient treated with the cancer immunotherapy is obtained. Most typically, the bodily fluid is selected from a group consisting of blood, serum, plasma, mucus, cerebrospinal fluid, and urine. From the patient bodily fluid, a quantity and a subtype of a cell free RNA of at least one cancer related gene is identified. Typically, the step of identifying the quantity includes amplifying a signal of cell free RNA by real time, quantitative RT-PCR. In some embodiments, the quantity of the cell free RNA is an indicative of immune response activation in the patient. Preferably, the subtype of a cell free RNA is associated with the type or location of tumors (e.g., neuroblastoma, non-small cell lung cancer, prostate cancer, etc.). Then, the quantity and the subtype of the cell free RNA is associated with the location of the tumor. In some embodiments, the step of associating comprises identification of an NK cell activation, identification of a T-cell mediated immune response activation, and identification of autophagy.

Preferably, the cancer immunotherapy is a treatment of the individual with a recombinant neoepitope vaccine, a treatment of the individual with an oncolytic virus, and/or a treatment of the individual with a checkpoint inhibitor. Also preferably, the cell free RNA is at least one of ctRNA or cfRNA. In some embodiments, the cell free RNA is mRNA encoding an inflammation-related protein. In other embodiments, the cell free RNA is mRNA encoding a protein selected from a group consisting of HMGB1, MUC1, VWF, MMP, CRP, PBEF1 TNF-α, TGF-β, PDGFA, and hTERT. Where the cell free RNA is mRNA encoding HMGB1, the mRNA encoding HMGB1 comprises a plurality of alternative splicing variants, and/or is generated in a cancer cell. Typically, the alternative splicing variants is cancer cell specific and/or tissue specific.

In some embodiments, the cell free RNA is a regulatory non-coding RNA. In such embodiments, expression of the regulatory non-coding RNA may modulate expression of mRNA encoding a protein selected from a group consisting of HMGB1, MUC1, VWF, MMP, CRP, PBEF1 TNF-α, TGF-β, PDGFA, and hTERT.

In some embodiments, the method further includes steps of obtaining a bodily fluid of a healthy individual, identifying a quantity and a subtype of the cell free RNA of the at least one cancer related gene in the bodily fluid of the healthy individual, and comparing the quantity and a subtype of the cell free RNA in the bodily fluid of the healthy individual with the quantity of the cell free RNA in the bodily fluid of the patient. In other embodiments, the method further includes steps of obtaining a bodily fluid of a patient before treating the patient with the cancer immunotherapy, identifying a pre-treatment quantity of the cell free RNA of the at least one cancer related gene in the bodily fluid of the patient, and comparing the pre-treatment quantity with the quantity of the cell free RNA in the bodily fluid of the patient.

In still another aspect of the inventive subject matter, the inventors contemplate a method for detecting autophagy in a patient treated with a cancer immunotherapy. In this method, a bodily fluid of a patient treated with the cancer immunotherapy is obtained. Most typically, the bodily fluid is selected from a group consisting of blood, serum, plasma, mucus, cerebrospinal fluid, and urine. In some embodiments, the bodily fluid of the patient is obtained at least 24 hours after the treatment with the cancer immunotherapy. From the patient bodily fluid, a quantity and a subtype of a cell free RNA of at least one autophagy related gene is identified. Typically, the step of identifying the quantity includes amplifying a signal of cell free RNA by real time, quantitative RT-PCR. Then, the quantity and the subtype of the cell free RNA is associated with a presence of autophagy in the patient.

Preferably, the cancer immunotherapy is a treatment of the individual with a recombinant neoepitope vaccine, a treatment of the individual with an oncolytic virus, and/or a treatment of the individual with a checkpoint inhibitor. Also preferably, the cell free RNA is at least one of ctRNA or cfRNA. In some embodiments, the cell free RNA is mRNA encoding an inflammation-related protein. In other embodiments, the cell free RNA is mRNA encoding a protein selected from a group consisting of HMGB1, MUC1, VWF, MMP, CRP, PBEF1 TNF-α, TGF-β, PDGFA, and hTERT. Where the cell free RNA is mRNA encoding HMGB1, the mRNA encoding HMGB1 comprises a plurality of alternative splicing variants, and/or is generated in a cancer cell and/or an immune cell. Typically, the alternative splicing variants is cancer cell specific and/or tissue specific.

In some embodiments, the cell free RNA is a regulatory non-coding RNA. In such embodiments, expression of the regulatory non-coding RNA may modulate expression of mRNA encoding a protein selected from a group consisting of HMGB1, MUC1, VWF, MMP, CRP, PBEF1 TNF-α, TGF-β, PDGFA, and hTERT.

In some embodiments, the method further comprise steps of obtaining a bodily fluid of a healthy individual, identifying a quantity of the cell free RNA of the at least one cancer related gene in the bodily fluid of the healthy individual, and comparing the quantity of the cell free RNA in the bodily fluid of the healthy individual with the quantity of the cell free RNA in the bodily fluid of the patient. In other embodiments, the method further comprise steps of obtaining a bodily fluid of a patient before treating the patient with the cancer immunotherapy, identifying a pre-treatment quantity of the cell free RNA of the at least one cancer related gene in the bodily fluid of the patient, and comparing the pre-treatment quantity with the quantity of the cell free RNA in the bodily fluid of the patient.

In still another aspect of the inventive subject matter, the inventors contemplate a method for identifying a compound effective to revert immune therapy resistant tumor cells to immune therapy sensitive tumor cells. In this method, a bodily fluid of a patient treated with the cancer immunotherapy and a compound is obtained. Most typically, the bodily fluid is selected from a group consisting of blood, serum, plasma, mucus, cerebrospinal fluid, and urine From the patient bodily fluid, a quantity and a subtype of a cell free RNA of at least one autophagy related gene is identified. Typically, the step of identifying the quantity includes amplifying a signal of cell free RNA by real time, quantitative RT-PCR. Then, the quantity and the subtype of the cell free RNA is associated with the effectiveness of the compound in reverting immune therapy resistant tumor cell to immune therapy sensitive tumor cell. Preferably, the step of associating comprises identification of an NK cell activation, identification of a T-cell mediated immune response activation, and identification of autophagy. In some embodiments, the quantity of the cell free RNA is an indicative of immune response activation in the patient. In some embodiments, the effectiveness includes a change in size or location of a tumor.

Preferably, the cancer immunotherapy is a treatment of the individual with a recombinant neoepitope vaccine, a treatment of the individual with an oncolytic virus, and/or a treatment of the individual with a checkpoint inhibitor. Also preferably, the cell free RNA is at least one of ctRNA or cfRNA. In some embodiments, the cell free RNA is mRNA encoding an inflammation-related protein. In other embodiments, the cell free RNA is mRNA encoding a protein selected from a group consisting of HMGB1, MUC1, VWF, MMP, CRP, PBEF1 TNF-α, TGF-β, PDGFA, and hTERT. Where the cell free RNA is mRNA encoding HMGB1, the mRNA encoding HMGB1 comprises a plurality of alternative splicing variants, and/or is generated in a cancer cell. Typically, the alternative splicing variants is cancer cell specific and/or tissue specific.

In some embodiments, the cell free RNA is a regulatory non-coding RNA. In such embodiments, expression of the regulatory non-coding RNA may modulate expression of mRNA encoding a protein selected from a group consisting of HMGB1, MUC1, VWF, MMP, CRP, PBEF1 TNF-α, TGF-β, PDGFA, and hTERT.

In some embodiments, the method further comprises steps of obtaining a bodily fluid of a patient before treating the patient with the cancer immunotherapy and the compound, identifying a pre-treatment quantity of the cell free RNA of the at least one cancer related gene in the bodily fluid of the patient, and comparing the pre-treatment quantity with the quantity of the cell free RNA in the bodily fluid of the patient. In other embodiments, the method further comprises steps of obtaining a bodily fluid of a patient before treating the patient with the compound and after treating the patient with the cancer immunotherapy, identifying a pre-treatment quantity of the cell free RNA of the at least one cancer related gene in the bodily fluid of the patient, and comparing the pre-treatment quantity with the quantity of the cell free RNA in the bodily fluid of the patient.

Additionally, the inventors also contemplate uses of cell free RNA to determine prognosis of a cancer immunotherapy, to identify a location of a tumor that is susceptible to a cancer immunotherapy, to detect autophagy after a cancer therapy, or to identify a compound effective to revert immune therapy resistant tumor cell to immune therapy sensitive tumor cell, using any of contemplated methods described above.

Most typically, the cell free RNA is at least one of ctRNA or cfRNA, which can be mRNA encoding an inflammation-related protein or a regulatory noncoding RNA. The inventors contemplated that the expression level of such cell free RNA is changed upon effective treatment of cancer immunotherapy. Thus, it is also contemplated that the expression level of the cell free RNA can be obtained from a healthy individual or from a patient before cancer immunotherapy so that the quantity of the cell free RNA in the bodily fluid of the cancer patient after treatment with cancer immunotherapy can be compared with the quantity of the cell free RNA in the bodily fluid of a healthy individual or a patient without the cancer immunotherapy.

Various objects, features, aspects and advantages of the inventive subject matter will become more apparent from the following detailed description of preferred embodiments.

### DETAILED DESCRIPTION

The inventors discovered that expression levels and/or the ratio of subtype(s) of certain cell free RNAs in a bodily fluid of a patient are modified when a tumor (especially malignant tumor) is present in the patient's body as compared to a healthy individual. The inventors further discovered that that the expression level and/or ratio of subtype(s) of such cell free RNA in the bodily fluid of the patient are modified when the patient is treated with a cancer immunotherapy. Thus, the inventors contemplate that such cell free RNA can be used as an indicator for assessing the prognosis of the cancer immunotherapy. In addition, the inventors also discovered that from analysis of the subtype and/or quantity of the cell free RNA in the bodily fluid of the patient, the location of the tumor and/or presence of autophagy of specific tumor that is targeted by the immunotherapy may be identified. Further, the inventors discovered that analysis of cell free RNA in a bodily fluid of a patient can be used to identify a compound that can revert immune therapy resistant tumor cell to immune therapy sensitive tumor cell.

As used herein, the term “tumor” refers to, and is interchangeably used with one or more cancer cells, cancer tissues, malignant tumor cells, or malignant tumor tissue, that can be placed or found in one or more anatomical locations in a human body. It should be noted that the term “patient” as used herein includes both individuals that are diagnosed with a condition (e.g., cancer) as well as individuals undergoing examination and/or testing for the purpose of detecting or identifying a condition. Thus, a patient having a tumor refers to both individuals that are diagnosed with a cancer as well as individuals that are suspected to have a cancer. As used herein, the term “provide” or “providing” refers to and includes any acts of manufacturing, generating, placing, enabling to use, transferring, or making ready to use.

### Cancer Immunotherapy

Any suitable cancer immunotherapy methods that are capable of eliciting systemic or local immune responses against a tumor, are deemed suitable for use herein. One exemplary cancer immunotherapy method employs a recombinant neoepitope vaccine. With respect to suitable neoepitopes, it should be appreciated that any epitope that is cancer associated, specific to a type of cancer, or a patient-specific neoepitope that is capable of triggering NK cell activation, T-cell mediated immune response, or autophagy, is suitable for use herein, particularly where the epitope is expressed (preferably above healthy control), and that further preferred epitopes include those predicted of binding to the respective binding motifs of the MHC-I and/or MHC-II complex as also further described in more detail below.

Neoepitopes can be characterized as expressed random mutations in tumor cells that created unique and tumor specific antigens. Therefore, viewed from a different perspective, neoepitopes may be identified by considering the type (e.g., deletion, insertion, transversion, transition, translocation) and impact of the mutation (e.g., non-sense, missense, frame shift, etc.), which may as such serve as a first content filter through which silent and other non-relevant (e.g., non-expressed) mutations are eliminated. It should further be appreciated that neoepitope sequences can be defined as sequence stretches with relatively short length (e.g., 7-11 mers) wherein such stretches will include the change(s) in the amino acid sequences. Most typically, the changed amino acid will be at or near the central amino acid position. For example, a typical neoepitope may have the structure of A₄-N-A₄, or A₃-N-A₅, or A₂-N-A₇, or A₅-N-A₃, or A₇-N-A₂, where A is a proteinogenic amino acid and N is a changed amino acid (relative to wild type or relative to matched normal). For example, neoepitope sequences as contemplated herein include sequence stretches with relatively short length (e.g., 5-30 mers, more typically 7-11 mers, or 12-25 mers) wherein such stretches include the change(s) in the amino acid sequences.

Neoepitopes may be identified from a patient tumor in a first step by whole genome analysis of a tumor biopsy (or lymph biopsy or biopsy of a metastatic site) and matched normal tissue (i.e., non-diseased tissue from the same patient) via synchronous comparison of the so obtained omics information. So identified neoepitopes can then be further filtered for a match to the patient's HLA type to increase likelihood of antigen presentation of the neoepitope. Most preferably and as further discussed below, such matching can be done in silico. Most typically, the patient-specific epitopes are unique to the patient, but may also in at least some cases include tumor type-specific neoepitopes (e.g., Her-2, PSA, brachyury, etc.) or cancer-associated neoepitopes (e.g., CEA, MUC-1, CYPB1, etc.). Thus, it should be appreciated that the recombinant nucleic acid construct (e.g., adenoviral expression construct for delivery by adenovirus) will include a recombinant segment that encodes at least one patient-specific neoepitope, and more typically encode at least two or three more neoepitopes and/or tumor type-specific neoepitopes and/or cancer-associated neoepitopes. Where the number of desirable neoepitopes is larger than the viral capacity for recombinant nucleic acids, multiple and distinct neoepitopes may be delivered via multiple and distinct recombinant viruses. Alternatively, nucleic acids may also be directly delivered to a cell via transfection or via DNA/RNA vaccine compositions.

Consequently, it should be recognized that patient and cancer specific neoepitopes can be identified in an exclusively in silico environment that ultimately predicts potential epitopes that are unique to the patient and tumor type. So identified and selected neoepitopes can then be further filtered in silico against an identified patient HLA-type. Such HLA-matching is thought to ensure strong binding of the neoepitopes to the MHC-I complex of nucleated cells and the MHC-II complex of specific antigen presenting cells. Targeting both antigen presentation systems is particularly thought to produce a therapeutically effective and durable immune response involving both, the cellular and the humoral branch of the immune system. Preferably, such identified neoepitopes are packaged in the recombinant nucleic acids, which then may be administered as a DNA vaccine, or further assembled into a viral genome so that the neoepitopes can be expressed when the virus infects the cancer cells.

For another example, the inventors contemplate that administering an oncolytic virus to the patients can effectively elicit NK cell immune response and/or T-cell mediated immune response without necessarily inducing oncolysis reactions. While any suitable type of oncolytic virus is contemplated (e.g., adenoviruses, poxviruses, HSV-1, coxsackieviruses, poliovirus, measles virus, and Newcastle disease virus (NDV)), it is especially preferred that the oncolytic viruses are genetically modified to present low immunogenicity to the host. For example, a preferred oncolytic virus includes genetically modified adenovirus serotype 5 (Ad5) with one or more deletions in its early 1 (E1), early 2b (E2b), or early 3 (E3) gene (e.g., E1 and E3 gene-deleted Ad5 (Ad5[E1]), E2b gene-deleted Ad5 (Ad5[E1,E2b], etc.). In one preferred virus strains having Ad5 [E1-, E2b-] vector platform, early 1 (E1), early 2b (E2b), and early 3 (E3) gene regions encoding viral proteins against which cell mediated immunity arises, are deleted to reduce immunogenicity. Also, in this strain, deletion of the Ad5 polymerase (pol) and preterminal protein (pTP) within the E2b region reduces Ad5 downstream gene expression which includes Ad5 late genes that encode highly immunogenic and potentially toxic proteins. Viewed from a different perspective and among other suitable viruses, particularly preferred oncolytic viruses include non-replicating or replication deficient adenoviruses that may be genetically engineered to trigger a stress response in an infected cell to thereby increase expression of NKG2D in the infected cell.

In some embodiments, the oncolytic virus can be modified to express additional proteins or inhibit intrinsic protein expression in the cancer cells may augment effectiveness of oncolytic virus for the NK cell activation. Most preferably, the oncolytic virus is genetically engineered to force an infected cell to express one or more stress signals that in turn trigger (an increased) expression of NKG2D. Thus, in some embodiments, a viral vector (e.g., recombinant adenovirus genome, optionally with a deleted or non-functional E2b gene) is genetically modified to include a nucleic acid that encodes, for example, a) NK cell receptor ligand (e.g., NKG2D ligands), b) modified HLA-E and/or hsp60, c) DNAM-1 ligand, d) a peptide that binds to a checkpoint receptor, e) regulatory elements (e.g., miRNA, shRNA, or siRNA) that reduce expression of at least one of MEW class 1 molecule, MEW class 2 molecule, TGF-b½ or a metalloproteinase, f) one or more secreted cytokine including GMCSF, FMS-related tyrosine kinase 3, CCL3, CCL5, TNF-a, IL-2, and IL-4. Most typically, wherein the nucleic acid encodes a membrane protein or secreted protein, the nucleotide will further include a trafficking signal to direct a peptide product encoded by the nucleic acid to the cytoplasm, the endosomal compartment, or the lysosomal compartment, and the peptide product will further comprise a sequence portion that enhances intracellular turnover of the peptide product. In these embodiments, the entry of oncolytic virus to the cancer cells not only cause cell stress to express NK cell activating signals, such NK cell activating signals will be augmented with further overexpression of NK cell ligands or downregulation of MHC molecules on the cell surface by genes encoding such proteins or regulatory elements.

In still another example, the inventors also contemplate that a patient can be administered a pharmaceutical compound that can trigger or augment the immune response against a cancer cell. Especially contemplated pharmaceutical compounds include immune checkpoint inhibitors that prevent interactions between tumor cells and immune cells. Immune checkpoint inhibitors can be administered to a patient alone or with recombinant neoepitope vaccine. With respect to suitable checkpoint inhibitors it is contemplated that all compounds and compositions that interfere with checkpoint signaling (e.g., CTLA-4 (CD152), PD-1 (CD 279), Tim-3, Lag-3, etc.) are deemed suitable for use herein. For example, particularly preferred checkpoint inhibitors include pembrolizumab, nivolumab, and ipilimumab. Most typically, checkpoint inhibitors will be administered following conventional protocol and as described in the prescription information. However, it should be noted that where the checkpoint inhibitors are peptides or proteins, such peptides and/or proteins can also be expressed in the patient from any suitable expression system (alone or in combination with neoepitopes and/or co-stimulatory molecules). Moreover, as used herein, the term ‘administering’ with respect to a checkpoint inhibitor refers to direct administration (e.g., by a physician or other licensed medical professional, etc.) or indirect administration (e.g., causing or advising to administer) of the checkpoint inhibitor to a patient.

With respect to dose, schedule and/or duration of the cancer immunotherapy, it is contemplated that the dose and/or schedule may vary depending on the tumor type, size, location, patient's health status (e.g., including age, gender, etc.), and any other relevant conditions. While it may vary, the dose and schedule may be selected and regulated so that the cancer immunotherapy does not provide any direct and significant toxic effect to the host normal cells, yet sufficient to be effective to activate patient's immune system against the tumors or trigger autophagy of the tumor cells. Thus, in a preferred embodiment, an optimal or desired condition of providing cancer immunotherapy that targets to activate NK cells or T-cell mediated immune response can be determined based on a predetermined threshold. For example, the predetermined threshold may be a predetermined rate of immediate lysis (e.g., within 1 hour after exposure to the cancer immunotherapy, within 6 hours after exposure to the cancer immunotherapy, etc.) of the tumor cells and/or nearby normal cells. Therefore, conditions are typically adjusted to have an immediate cell killing effect on less than 50%, and more typically less than 30%, even more typically less than 10%, and most typically less than 5% of all cells in the tissue. For example, where the cancer immunotherapy is administration of oncolytic virus, the tumor cells infected by oncolytic virus will be viable at least for a period of time enough to preferably exhibit a substantially altered gene expression or protein expression profile due to induced cell stress by the oncolytic virus.

In another embodiment, where the cancer immunotherapy targets trigger autophagy of the cancer cell, an optimal or desired condition of providing the cancer therapy can be determined by the quantity of cells undergoing autophagy (e.g., determined by tissue biopsy, etc.). Therefore, conditions are typically adjusted to have an autophagy rate of the cancer cell on more than 5%, and more typically more than 10%, even more typically more than 20%, and most typically more than 30% of all cancer cells in the tissue within less than 1 day, less than 7 days, or less than 2 weeks from the initiation of the cancer immunotherapy.

### Cell-Free RNA

The inventors contemplate that treatment of a cancer patient with one or more cancer immunotherapy can trigger release of cell free nucleic acid, preferably cell free RNA to the patient's bodily fluid, thus increase the quantity of the cell free RNA. As used herein, the patient's bodily fluid includes, but is not limited to, blood, serum, plasma, mucus, cerebrospinal fluid, ascites fluid, saliva, and urine of the patient. The patient's bodily fluid may be fresh or preserved/frozen.

The cell free RNA may include any types of RNA that are circulating in the bodily fluid of a person without being enclosed in a cell body or a nucleus. Most typically, the source of the cell free RNA is the cell directly or indirectly affected by the cancer immunotherapy, preferably a cancer cell. However, it is also contemplated that the source of the cell free RNA is the immune cell (e.g., NK cells, T cells, macrophages, etc.). Thus, the cell free RNA can be circulating tumor RNA (ctRNA) and/or circulating free RNA (cfRNA, circulating nucleic acids that do not derive from a tumor). While not wishing to be bound by a particular theory, it is contemplated that the release of cell free RNA originated from the tumor cell can be increased when the tumor cell interact with the immune cell or when the tumor cells undergo cell death (e.g., necrosis, apoptosis, autophagy, etc.). Thus, in some embodiments, the cell free RNA may be enclosed in a vesicular structure (e.g., via exosomal release of cytoplasmic substances) so that it can be protected from RNase activity in some type of bodily fluid. Yet, it is also contemplated that in other embodiments, the cell free RNA is a naked RNA without being enclosed in any membranous structure, but may be stabilized via interaction with non-nucleotide molecules (e.g., any RNA binding proteins, etc.).

Therefore, in addition to quantification of HMBG cell free RNA as described in more detail below, it is contemplated that the methods presented herein will also include quantification of total cell free RNA and/or specific fractions thereof to determine the presence of or absence of cancer in the patient. Where specific fractions are quantified, it should be appreciated that such fractions may be particularly relevant to the specific disease. For example, especially suitable RNA fractions include those representing tumor associated genes and/or neoepitopes specific to a tumor in the patient. Alternatively and/or additionally, circulating RNA encoding DNA repair genes are also deemed suitable. As will be readily appreciated, such additional measurements may be used as a baseline and/or as an indicator of treatment efficacy. Examples for suitable methods are disclosed in co-pending U.S. provisional applications 62/504,149, filed May 10, 2017, 62/473,273, filed Mar. 17, 2017, and 62/500,497 filed May 3, 2017, all incorporated by reference herein.

It is contemplated that the cell free RNA can be any type of RNA which can be released from either cancer cells or immune cell. Thus, the cell free RNA may include mRNA, tRNA, microRNA, small interfering RNA, long non-coding RNA (lncRNA). Most typically, the cell free RNA is a full length or a fragment of mRNA (e.g., at least 70% of full-length, at least 50% of full length, at least 30% of full length, etc.) encoding one or more cancer-related proteins, or inflammation-related proteins. For example, the cell free mRNA are derived from the cancer related gene including, but not limited to, ABL1, ABL2, ACTB, ACVR1B, AKT1, AKT2, AKT3, ALK, AMER11, APC, AR, ARAF, ARFRP1, ARID1A, ARID1B, ASXL1, ATF1, ATM, ATR, ATRX, AURKA, AURKB, AXIN1, AXL, BAP1, BARD1, BCL2, BCL2L1, BCL2L2, BCL6, BCOR, BCORL1, BLM, BMPR1A, BRAF, BRCA1, BRCA2, BRD4, BRIP1, BTG1, BTK, EMSY, CARD11, CBFB, CBL, CCND1, CCND2, CCND3, CCNE1, CD274, CD79A, CD79B, CDC73, CDH1, CDK12, CDK4, CDK6, CDK8, CDKN1A, CDKN1B, CDKN2A, CDKN2B, CDKN2C, CEA, CEBPA, CHD2, CHD4, CHEK1, CHEK2, CIC, CREBBP, CRKL, CRLF2, CSF1R, CTCF, CTLA4, CTNNA1, CTNNB1, CUL3, CYLD, DAXX, DDR2, DEPTOR, DICER1, DNMT3A, DOT1L, EGFR, EP300, EPCAM, EPHA3, EPHA5, EPHA7, EPHB1, ERBB2, ERBB3, ERBB4, EREG, ERG, ERRFIl, ESR1, EWSR1, EZH2, FAM46C, FANCA, FANCC, FANCD2, FANCE, FANCF, FANCG, FANCL, FAS, FAT1, FBXW7, FGF10, FGF14, FGF19, FGF23, FGF3, FGF4, FGF6, FGFR1, FGFR2, FGFR3, FGFR4, FH, FLCN, FLI1, FLT1, FLT3, FLT4, FOLH1, FOXL2, FOXP1, FRS2, FUBP1, GABRA6, GATA1, GATA2, GATA3, GATA4, GATA6, GID4, GLI1, GNA11, GNA13, GNAQ, GNAS, GPR124, GRIN2A, GRM3, GSK3B, H3F3A, HAVCR2, HGF, HMGB1, HMGB2, HMGB3, HNF1A, HRAS, HSD3B1, HSP90AA1, IDHL IDH2, IDO, IGF1R, IGF2, IKBKE, IKZF1, IL7R, INHBA, INPP4B, IRF2, IRF4, IRS2, JAK1, JAK2, JAK3, JUN, MYST3, KDM5A, KDM5C, KDM6A, KDR, KEAP, KEL, KIT, KLHL6, KLK3, MLL, MLL2, MLL3, KRAS, LAG3, LMO1, LRP1B, LYN, LZTR1, MAGI2, MAP2K1, MAP2K2, MAP2K4, MAP3K1, MCL1, MDM2, MDM4, MED12, MEF2B, MEN1, MET, MITF, MLH1, MPL, MRE11A, MSH2, MSH6, MTOR, MUC1, MUTYH, MYC, MYCL, MYCN, MYD88, MYH, NF1, NF2, NFE2L2, NFKB1A, NKX2-1, NOTCH1, NOTCH2, NOTCH3, NPM1, NRAS, NSD1, NTRK1, NTRK2, NTRK3, NUP93, PAK3, PALB2, PARK2, PAX3, PAX, PBRM1, PDGFRA, PDCD1, PDCD1LG2, PDGFRB, PDK1, PGR, PIK3C2B, PIK3CA, PIK3CB, PIK3CG, PIK3R1, PIK3R2, PLCG2, PMS2, POLD1, POLE, PPP2R1A, PREX2, PRKAR1A, PRKC1, PRKDC, PRS S8, PTCH1, PTEN, PTPN11, QK1, RAC1, RAD50, RAD51, RAF1, RANBP1, RARA, RB1, RBM10, RET, RICTOR, RIT1, RNF43, ROS1, RPTOR, RUNX1, RUNX1T1, SDHA, SDHB, SDHC, SDHD, SETD2, SF3B1, SLIT2, SMAD2, SMAD3, SMAD4, SMARCA4, SMARCB1, SMO, SNCAIP, SOCS1, SOX10, SOX2, SOX9, SPEN, SPOP, SPTA1, SRC, STAG2, STAT3, STAT4, STK11, SUFU, SYK, T (BRACHYURY), TAF1, TBX3, TERC, TERT, TET2, TGFRB2, TNFAIP3, TNFRSF14, TOP1, TOP2A, TP53, TSC1, TSC2, TSHR, U2AF1, VEGFA, VHL, WISP3, WT1, XPO1, ZBTB2, ZNF217, ZNF703, CD26, CD49F, CD44, CD49F, CD13, CD15, CD29, CD151, CD138, CD166, CD133, CD45, CD90, CD24, CD44, CD38, CD47, CD96, CD 45, CD90, ABCBS, ABCG2, ALCAM, ALPHA-FETOPROTEIN, DLL1, DLL3, DLL4, ENDOGLIN, GJA1, OVASTACIN, AMACR, NESTIN, STRO-1, MICL, ALDH, BMI-1, GLI-2, CXCR1, CXCR2, CX3CR1, CX3CL1, CXCR4, PON1, TROP1, LGR5, MSI-1, C-MAF, TNFRSF7, TNFRSF16, SOX2, PODOPLANIN, L1CAM, HIF-2 ALPHA, TFRC, ERCC1, TUBB3, TOP1, TOP2A, TOP2B, ENOX2, TYMP, TYMS, FOLR1, GPNMB, PAPPA, GART, EBNA1, EBNA2, LMP1, BAGE, BAGE2, BCMA, C10ORF54, CD4, CD8, CD19, CD20, CD25, CD30, CD33, CD80, CD86, CD123, CD276, CCL1, CCL2, CCL3, CCL4, CCL5, CCL7, CCL8, CCL11, CCL13, CCL14, CCL15, CCL16, CCL17, CCL18, CCL19, CCL20, CCL21, CCL22, CCL23, CCL24, CCL25, CCL26, CCL27, CCL28, CCR1, CCR2, CCR3, CCR4, CCR5, CCR6, CCR7, CCR8, CCR9, CCR10, CXCL1, CXCL2, CXCL3, CXCL5, CXCL6, CXCL9, CXCL10, CXCL11, CXCL12, CXCL13, CXCL14, CXCL16, CXCL17, CXCR3, CXCR5, CXCR6, CTAG1B, CTAG2, CTAG1, CTAG4, CTAG5, CTAG6, CTAG9, CAGE1, GAGE1, GAGE2A, GAGE2B, GAGE2C, GAGE2D, GAGE2E, GAGE4, GAGE10, GAGE12D, GAGE12F, GAGE12J, GAGE13, HHLA2, ICOSLG, LAG1, MAGEA10, MAGEA12, MAGEA1, MAGEA2, MAGEA3, MAGEA4, MAGEA4, MAGEA5, MAGEA6, MAGEA7, MAGEA8, MAGEA9, MAGEB1, MAGEB2, MAGEB3, MAGEB4, MAGEB6, MAGEB10, MAGEB16, MAGEB18, MAGEC1, MAGEC2, MAGEC3, MAGED1, MAGED2, MAGED4, MAGED4B, MAGEE1, MAGEE2, MAGEF1, MAGEH1, MAGEL2, NCR3LG1, SLAMF7, SPAG1, SPAG4, SPAG5, SPAG6, SPAG7, SPAG8, SPAG9, SPAG11A, SPAG11B, SPAG16, SPAG17, VTCN1, XAGE1D, XAGE2, XAGE3, XAGE5, XCL1, XCL2, and XCR1. Of course, it should be appreciated that the above genes may be wild type or mutated versions, including missense or nonsense mutations, insertions, deletions, fusions, and/or translocations, all of which may or may not cause formation of full-length mRNA.

For another example, the cell free mRNA are those encoding a full length or a fragment of inflammation-related proteins, including, but not limited to, HMGB1, HMGB2, HMGB3, MUC1, VWF, MMP, CRP, PBEF1, TNF-α, TGF-β, PDGFA, IL-1, IL-2, IL-3, IL-4, IL-5, IL-6, IL-7, IL-8, IL-9, IL-10, IL-12, IL-13, IL-15, IL-17, Eotaxin, FGF, G-CSF, GM-CSF, IFN-γ, IP-10, MCP-1, PDGF, and hTERT, and in yet another example, the cell free mRNA encoded a full length or a fragment of HMGB1.

The cell free mRNA may be present in a plurality of isoforms (e.g., splicing variants, etc.) that may be associated with different cell types and/or location. Preferably, different isoforms of mRNA may be a hallmark of specific tissues (e.g., brain, intestine, adipose tissue, muscle, etc.), or may be a hallmark of cancer (e.g., different isoform is present in the cancer cell compared to corresponding normal cell, or the ratio of different isoforms is different in the cancer cell compared to corresponding normal cell, etc.). For example, mRNA encoding HMGB1 are present in 18 different alternative splicing variants and 2 unspliced forms. Those isoforms are expected to express in different tissues/locations of the patient's body (e.g., isoform A is specific to prostate, isoform B is specific to brain, isoform C is specific to spleen, etc.). Thus, in these embodiments, identifying the isoforms of cell free mRNA in the patient's bodily fluid can provide information on the origin (e.g., cell type, tissue type, etc.) of the cell free mRNA.

The inventors contemplate that the quantities and/or isoforms (or subtypes) or regulatory noncoding RNA (e.g., microRNA, small interfering RNA, long non-coding RNA (lncRNA)) can vary and fluctuate by presence of a tumor or immune response against the tumor. Without wishing to be bound by any specific theory, varied expression of regulatory noncoding RNA in a cancer patient's bodily fluid may due to genetic modification of the cancer cell (e.g., deletion, translocation of parts of a chromosome, etc.), and/or inflammations at the cancer tissue by immune system (e.g., regulation of miR-29 family by activation of interferon signaling and/or virus infection, etc.). Thus, in some embodiments, the cell free RNA can be a regulatory noncoding RNA that modulates expression (e.g., downregulates, silences, etc.) of mRNA encoding a cancer-related protein or an inflammation-related protein (e.g., HMGB1, HMGB2, HMGB3, MUC1, VWF, MMP, CRP, PBEF1, TNF-α, TGF-β, PDGFA, IL-1, IL-2, IL-3, IL-4, IL-5, IL-6, IL-7, IL-8, IL-9, IL-10, IL-12, IL-13, IL-15, IL-17, Eotaxin, FGF, G-CSF, GM-CSF, IFN-γ, IP-10, MCP-1, PDGF, hTERT, etc.).

It is also contemplated that some cell free regulatory noncoding RNA may be present in a plurality of isoforms or members (e.g., members of miR-29 family, etc.) that may be associated with different cell types and/or location. Preferably, different isoforms or members of regulatory noncoding RNA may be a hallmark of specific tissues (e.g., brain, intestine, adipose tissue, muscle, etc.), or may be a hallmark of cancer (e.g., different isoform is present in the cancer cell compared to corresponding normal cell, or the ratio of different isoforms is different in the cancer cell compared to corresponding normal cell, etc.). For example, higher expression level of miR-155 in the bodily fluid can be associated with the presence of breast tumor, and the reduced expression level of miR-155 can be associated with reduced size of breast tumor. Thus, in these embodiments, identifying the isoforms of cell free regulatory noncoding RNA in the patient's bodily fluid can provide information on the origin (e.g., cell type, tissue type, etc.) of the cell free regulatory noncoding RNA.

Without wishing to be bound by any specific theory, the inventors contemplate that detection of type and/or expression level of cell free RNA(s) may provide a more sensitive indication of the modulation of the corresponding proteins compared to detection of serum level of the corresponding protein. For example, HMGB1 protein exist in various forms in various subcellular or cellular locations, including hyper-acetylated form in the cytosol, a normal form in the nucleus, a secreted form in the macrophage and/or dendritic cells. Thus, detection of one or more types of HMGB1 protein in the serum may not reflect the overall changes of the HMGB1 protein in the tumor tissue. Rather, the inventors contemplate that detection of one or more types of mRNA(s) (that may or may not be associated with the prognosis of the disease, effectiveness of the immune therapy, etc.) of HMGB1 (with or without concurrent or subsequent measurement of protein expression level of HMGB1 in the serum) may provide more accurate assessments of the type-specific (e.g., with mutation, alternative splicing form, with or without a signaling sequence, etc.), cell-specific, subcellular location-specific, and/or overall expression levels of HMGB1 in the tumor tissue.

### Isolation and Amplification of Cell Free RNA

Any suitable methods to isolate and amplify cell free RNA are contemplated. Most typically, cell free RNA is isolated from a bodily fluid (e.g., whole blood) that is processed under conditions that stabilize cell free mRNA. Once separated from the non-nucleic acid components, cell free RNA are then quantified, preferably using real time, quantitative RT-PCR.

The bodily fluid of the patient can be obtained at any desired time point(s) depending on the purpose of the omics analysis. For example, the bodily fluid of the patient can be obtained before and/or after the patient is confirmed to have a tumor and/or periodically thereafter (e.g., every week, every month, etc.) in order to associate the cfRNA data with the prognosis of the cancer. In some embodiments, the bodily fluid of the patient can be obtained from a patient before and after the cancer treatment (e.g., chemotherapy, radiotherapy, drug treatment, cancer immunotherapy, etc.). While it may vary depending on the type of treatments and/or the type of cancer, the bodily fluid of the patient can be obtained at least 24 hours, at least 3 days, at least 7 days after the cancer treatment. For more accurate comparison, the bodily fluid from the patient before the cancer treatment can be obtained less than 1 hour, less than 6 hours before, less than 24 hours before, less than a week before the beginning of the cancer treatment. In addition, a plurality of samples of the bodily fluid of the patient can be obtained during a period before and/or after the cancer treatment (e.g., once a day after 24 hours for 7 days, etc.).

Additionally or alternatively, the bodily fluid of a healthy individual can be obtained to compare the quantity and/or subtype expression of cell free RNA. As used herein, a healthy individual refers an individual without a tumor. Preferably, the healthy individual can be chosen among group of people shares characteristics with the patient (e.g., age, gender, ethnicity, diet, living environment, family history, etc.).

In more detail, suitable tissue sources include whole blood, which is preferably provided as plasma or serum. Alternatively, it should be noted that various other bodily fluids are also deemed appropriate so long as cell free RNA is present in such fluids. Appropriate fluids include saliva, ascites fluid, spinal fluid, urine, etc., which may be fresh or preserved/frozen. For example, for the analyses presented herein, specimens were accepted as 10 ml of whole blood drawn into cell-free RNA BCT® tubes or cell-free DNA BCT® tubes containing RNA stabilizers, respectively. Advantageously, cell free RNA is stable in whole blood in the cell-free RNA BCT tubes for seven days while cell free RNA is stable in whole blood in the cell-free DNA BCT Tubes for fourteen days, allowing time for shipping of patient samples from world-wide locations without the degradation of cell free RNA. Moreover, it is generally preferred that the cell free RNA is isolated using RNA stabilization agents that will not or substantially not (e.g., equal or less than 1%, or equal or less than 0.1%, or equal or less than 0.01%, or equal or less than 0.001%) lyse blood cells. Viewed from a different perspective, the RNA stabilization reagents will not lead to a substantial increase (e.g., increase in total RNA no more than 10%, or no more than 5%, or no more than 2%, or no more than 1%) in RNA quantities in serum or plasma after the reagents are combined with blood. Likewise, these reagents will also preserve physical integrity of the cells in the blood to reduce or even eliminate release of cellular RNA found in blood cell. Such preservation may be in form of collected blood that may or may not have been separated. In less preferred aspects, contemplated reagents will stabilize cell free RNA in a collected tissue other than blood for at 2 days, more preferably at least 5 days, and most preferably at least 7 days. Of course, it should be recognized that numerous other collection modalities are also deemed appropriate, and that the cell free RNA can be at least partially purified or adsorbed to a solid phase to so increase stability prior to further processing.

It is generally preferred that the cfRNA is isolated using RNA stabilization reagents. While any suitable RNA stabilization agents are contemplated, preferred RNA stabilization reagents include one or more of a nuclease inhibitor, a preservative agent, a metabolic inhibitor, and/or a chelator. For example, contemplated nuclease inhibitors may include RNAase inhibitors such as diethyl pyrocarbonate, ethanol, aurintricarboxylic acid (ATA), formamide, vanadyl-ribonucleoside complexes, macaloid, heparin, bentonite, ammonium sulfate, dithiothreitol (DTT), beta-mercaptoethanol, dithioerythritol, tris(2-carboxyethyl)phosphene hydrochloride, most typically in an amount of between 0.5 to 2.5 wt %. Preservative agents may include diazolidinyl urea (DU), imidazolidinyl urea, dimethoylol-5,5-dimethylhydantoin, dimethylol urea, 2-bromo-2-nitropropane-1,3-diol, oxazolidines, sodium hydroxymethyl glycinate, 5-hydroxymethoxymethyl-1-laza-3,7-dioxabicyclo[3.3.0]octane, 5-hydroxymethyl-1-laza-3,7dioxabicyclo[3.3.0]octane, 5-hydroxypoly[methyleneoxy]methyl-1-laza-3,7-dioxabicyclo[3.3.0]octane, quaternary adamantine or any combination thereof. In most examples, the preservative agent will be present in an amount of about 5-30 wt %. Moreover, it is generally contemplated that the preservative agents are free of chaotropic agents and/or detergents to reduce or avoid lysis of cells in contact with the preservative agents.

Suitable metabolic inhibitors may include glyceraldehyde, dihydroxyacetone phosphate, glyceraldehyde 3-phosphate, 1,3-bisphosphoglycerate, 3-phosphoglycerate, phosphoenolpyruvate, pyruvate, and glycerate dihydroxyacetate, and sodium fluoride, which concentration is typically in the range of between 0.1-10 wt %. Preferred chelators may include chelators of divalent cations, for example, ethylenediaminetetraacetic acid (EDTA) and/or ethylene glycol-bis(β-aminoethyl ether)-N,N,N′,N′-tetraacetic acid (EGTA), which concentration is typically in the range of between 1-15 wt %.

Additionally, RNA stabilizing reagent may further include protease inhibitors, phosphatase inhibitors and/or polyamines. Therefore, exemplary compositions for collecting and stabilizing ctRNA in whole blood may include aurintricarboxylic acid, diazolidinyl urea, glyceraldehyde/sodium fluoride, and/or EDTA. Further compositions and methods for ctRNA isolation are described in U.S. Pat. Nos. 8,304,187 and 8,586,306, which are incorporated by reference herein.

Most preferably, such contemplated RNA stabilization agents for ctRNA stabilization are disposed within a test tube that is suitable for blood collection, storage, transport, and/or centrifugation. Therefore, in most typical aspects, the collection tube is configured as an evacuated blood collection tube that also includes one or more serum separator substance to assist in separation of whole blood into a cell containing and a substantially cell free phase (no more than 1% of all cells present). In general, it is preferred that the RNA stabilization agents do not or substantially do not (e.g., equal or less than 1%, or equal or less than 0.1%, or equal or less than 0.01%, or equal or less than 0.001%, etc.) lyse blood cells. Viewed from a different perspective, RNA stabilization reagents will not lead to a substantial increase (e.g., increase in total RNA no more than 10%, or no more than 5%, or no more than 2%, or no more than 1%) in RNA quantities in serum or plasma after the reagents are combined with blood. Likewise, these reagents will also preserve physical integrity of the cells in the blood to reduce or even eliminate release of cellular RNA found in blood cell. Such preservation may be in form of collected blood that may or may not have been separated. In some aspects, contemplated reagents will stabilize cell free RNA in a collected tissue other than blood for at 2 days, more preferably at least 5 days, and most preferably at least 7 days. Of course, it should be recognized that numerous other collection modalities other than collection tube (e.g., a test plate, a chip, a collection paper, a cartridge, etc.) are also deemed appropriate, and that the cell free RNA can be at least partially purified or adsorbed to a solid phase to so increase stability prior to further processing.

As will be readily appreciated, fractionation of plasma and extraction of cell free RNA can be done in numerous manners. In one exemplary preferred aspect, whole blood in 10 mL tubes is centrifuged to fractionate plasma at 1600 rcf for 20 minutes. The so obtained plasma is then separated and centrifuged at 16,000 rcf for 10 minutes to remove cell debris. Of course, various alternative centrifugal protocols are also deemed suitable so long as the centrifugation will not lead to substantial cell lysis (e.g., lysis of no more than 1%, or no more than 0.1%, or no more than 0.01%, or no more than 0.001% of all cells). Cell free RNA is extracted from 2 mL of plasma using Qiagen reagents. For example, where cfRNA was isolated, the inventors used a second container that included a DNase that was retained in a filter material. Notably, the cell free RNA also included miRNA (and other regulatory RNA such as shRNA, siRNA, and intronic RNA). Therefore, it should be appreciated that contemplated compositions and methods are also suitable for analysis of miRNA and other RNAs from whole blood.

Moreover, it should also be recognized that the extraction protocol was designed to remove potential contaminating blood cells, other impurities, and maintain stability of the nucleic acids during the extraction. All nucleic acids were kept in bar-coded matrix storage tubes, with DNA stored at −4° C. and RNA stored at −80° C. or reverse-transcribed to cDNA that is then stored at −4° C. Notably, so isolated cell free RNA can be frozen prior to further processing.

Once cell free RNA is isolated, various types of omics data can be obtained using any suitable methods. With respect to RNA sequence data it should be noted that contemplated RNA sequence data include mRNA sequence data, splice variant data, polyadenylation information, etc. Moreover, it is generally preferred that the RNA sequence data also include a metric for the transcription strength (e.g., number of transcripts of a damage repair gene per million total transcripts, number of transcripts of a damage repair gene per total number of transcripts for all damage repair genes, number of transcripts of a damage repair gene per number of transcripts for actin or other household gene RNA, etc.), and for the transcript stability (e.g., a length of poly A tail, etc.).

With respect to the transcription strength (expression level), transcription strength of the cell free RNA can be examined by quantifying the cell free RNA. Quantification of cfRNA can be performed in numerous manners, however, expression of analytes is preferably measured by quantitative real-time RT-PCR of cfRNA using primers specific for each gene. For example, amplification can be performed using an assay in a 10 μL reaction mix containing 2 μL cfRNA, primers, and probe. mRNA of α-actin or β-actin can be used as an internal control for the input level of cfRNA. A standard curve of samples with known concentrations of each analyte was included in each PCR plate as well as positive and negative controls for each gene. Test samples were identified by scanning the 2D barcode on the matrix tubes containing the nucleic acids. Delta Ct (dCT) was calculated from the Ct value derived from quantitative PCR (qPCR) amplification for each analyte subtracted by the Ct value of actin for each individual patient's blood sample. Relative expression of patient specimens is calculated using a standard curve of delta Cts of serial dilutions of Universal Human Reference RNA or another control known to express the gene of interest set at a gene expression value of 10 or a suitable whole number allowing for a range of patient sample results for the specific to be resulted in the range of approximately 1 to 1000 (when the delta CTs were plotted against the log concentration of each analyte), preferably approximately 10. Alternatively and/or additionally, Delta Cts vs. log₁₀Relative Gene Expression (standard curves) for each gene test can be captured over hundreds of PCR plates of reactions (historical reactions). A linear regression analysis can be performed for each assays and used to calculate gene expression from a single point from the original standard curve going forward.

Alternatively or additionally, where discovery or scanning for new mutations or changes in expression of a particular gene is desired, real time quantitative PCR may be replaced by or added with RNAseq to so cover at least part of a patient transcriptome. Moreover, it should be appreciated that analysis can be performed static or over a time course with repeated sampling to obtain a dynamic picture without the need for biopsy of the tumor or a metastasis. Thus, in addition to RNA quantification, RNA sequencing of the cell free RNA (directly or via reverse transcription) may be performed to verify identity and/or identify post-transcriptional modifications, splice variations, and/or RNA editing. To that end, sequence information may be compared to prior RNA sequences of the same patient (of another patient, or a reference RNA), preferably using synchronous location guided analysis (e.g., using BAMBAM as described in US Pat. Pub. No. 2012/0059670 and/or US2012/0066001, etc.). Such analysis is particularly advantageous as such identified mutations can be filtered for neoepitopes that are unique to the patient, presented in the MHC I and/or II complex of the patient, and as such serve as therapeutic target. Moreover, suitable mutations may also be further characterized using a pathway model and the patient- and tumor-specific mutation to infer a physiological parameter of the tumor. For example, especially suitable pathway models include PARADIGM (see e.g., WO 2011/139345, WO 2013/062505) and similar models (see e.g., WO 2017/033154). Moreover, suitable mutations may also be unique to a sub-population of cancer cells. Thus, mutations may be selected based on the patient and specific tumor (and even metastasis), on the suitability as therapeutic target, type of gene (e.g., cancer driver gene), and affected function of the gene product encoded by the gene with the mutation.

Moreover, the inventors contemplate that multiple types of cell free RNA can be isolated, detected and/or quantified from the same bodily fluid sample of the patient such that the relationship or association among the mutation, quantity, and/or subtypes of cell free RNA can be determined for further analysis. Thus, in one embodiment, from a single bodily fluid sample or from a plurality of bodily fluid samples obtained in a substantially similar time points, from a patient, multiple cell free RNA species can be detected and quantified. In this embodiment, it is especially preferred that at least some of the cfRNA measurements are specific with respect to a cancer associated nucleic acid.

### Exemplary Use of Cell Free RNA

The inventors contemplate that identified quantity and/or subtype of cell free RNA in a patient can be used to monitoring of prognosis of the tumor, monitoring the effectiveness of treatment provided to the patients, determining a prognosis of a cancer immunotherapy evaluating a treatment regime based on a likelihood of success of the treatment regime, and even as discovery tool that allows repeated and non-invasive sampling of a patient. As used herein, the prognosis refers any indication and/or sign of disease progression, prediction of disease progression, or likely outcome of a treatment.

For example, patient A suffering from prostate cancer is treated with the first stage of cancer immunotherapy using oncolytic virus. Patient A's blood serum can be obtained 24 hours before, 24 hours after, and 3 days after the oncolytic virus treatment. Additionally, a blood serum from a healthy individual who is in the same age range of the patient A could be obtained for further comparison. From the patient A's blood serum, cell free RNA was purified and amplified by real time RT-PCR, using random primers (to amplify substantially all cell free RNA) or gene-specific primers (to amplify RNA of specific gene). Then amplified cell free RNA(s) are quantified and characterized. As an exemplary result, the expression level of subtype A HMGB1 mRNA (specific to prostate cells) is increased for 30% compared to other subtypes of HMGB1 in 24 hours after the oncolytic virus treatment, and further increased for 50% in 3 days after the oncolytic virus treatment compared to the subtype A HMGB1 mRNA expression level of the patient before the oncolytic virus treatment. The similar increase could be found compared to the subtype A HMGB1 mRNA expression level of the healthy individual.

Such quantitative and qualitative analysis can be associated with the prognosis of the cancer immunotherapy and/or effectiveness of the oncolytic virus treatment. For example, the increase of subtype A HMGB1 mRNA expression can be an indicator of increased cell death of prostate cancer tissue, which further indicates that the oncolytic virus treatment was effective enough to increase the cancer cell death (either by autophagy or necrosis) by oncolytic virus infection, which may contribute less growth or even remission of tumor tissue in the prostate.

Additionally, if the result provide that another subtype of HMGB1 mRNA (specific to immune cells such as T cells or NK cells) is increased as well during the same period, the increase of those subtypes of HMGB1 mRNA expressions can be an indicator of increased cell death of prostate cancer tissue by activation of immune response triggered by oncolytic virus infection. Further, as the expression level of subtype A HMGB1 mRNA kept increasing over time to 3 days, it provides guidance that monitoring of subtype A HMGB1 mRNA would be required for next couple more days. In addition, such analysis can contribute to provide a future treatment plan of multiple, repeated oncolytic virus treatment to the prostate cancer patient to boost the effect.

Notably, use of the HMGB1 protein has been reported as an indicator of autophagy (see e.g., Molecular Therapy (2013) Vol. 21 no. 6, 1212-1223), however, numerous problems are associated with the use of the HMGB1 protein. Among other things, HMGB1 protein occurs in numerous posttranslational modified forms and even in different splice variants, depending on the particular origin of the protein. As such, the source of any measured (if at all measurable) protein is not clearly traceable to the tumor.

The inventors also contemplate that identified quantity and/or subtype of cell free RNA in a patient can advantageously also be used to identify a location of a tumor that is susceptible to a cancer immunotherapy. For example, patient A suffering from multiple type of tumors (e.g., brain tumors and lung cancer, either independent cancer or metastasized tumors) is treated with the first stage of cancer immunotherapy using oncolytic virus. Patient A's blood serum can be obtained 24 hours before, 24 hours after, and 3 days after the oncolytic virus treatment. Additionally, a blood serum from a healthy individual who is in the same age range of the patient A could be obtained for further comparison. From the patient A's blood serum, cell free RNA was purified and amplified by real time RT-PCR, using random primers (to amplify substantially all cell free RNA) or gene-specific primers (to amplify RNA of specific gene). Then amplified cell free RNA(s) are quantified and characterized. As an exemplary result, the expression level of subtype B HMGB1 mRNA (specific to brain) is increased for 30% compared to other subtypes of HMGB1 in 24 hours after the oncolytic virus treatment, and further increased for 50% in 3 days after the oncolytic virus treatment compared to the subtype B HMGB1 mRNA expression level of the patient before the oncolytic virus treatment. The similar increase could be found compared to the subtype B HMGB1 mRNA expression level of the healthy individual. In addition, no change in expression level of any other subtype of HMGB1 is observed.

Such quantitative and qualitative analysis can be associated with the location of the tumors that are susceptible to and/or more effectively treatable by the oncolytic virus treatment. For example, the specific increase of subtype B HMGB1 mRNA expression can be an indicator of increased cell death of brain tumor cells, which further indicates that the oncolytic virus treatment was effective enough to increase the cancer cell death (either by autophagy or necrosis) in the brain tumor, but not so effective in the lung cancer cells, by oncolytic virus infection.

Thus, the inventors further contemplate that the same method can be used to detect autophagy in a patient treated with a cancer immunotherapy. Tumor cells typically show increased autophagy after anti-cancer treatment (e.g., chemotherapy, radiotherapy, etc.). During the autophagy process, several cell free RNAs may be released from the dying tumor cells, and the presence and/or increased expression of such cell free RNAs can be an indicator of the presence of the autophagy in the patient's body.

Additionally, the identified quantity and/or subtype of cell free RNA in a patient can be used to identify a compound effective to revert cancer immunotherapy resistant tumor cell to cancer immunotherapy sensitive tumor cell. In preferred aspects, levels of HMGB1 cell free RNA can be positively correlated with likely positive treatment outcome. Any compound that can be administered to a patient concurrently or sequentially with cancer immunotherapy is contemplated. For example, any compounds that can target the mutated or altered (e.g., over- or under-expressed, redistributed, cleaved and released, altered post-translational modification, etc.) element (e.g., genes, mRNA, protein, miRNA, etc.) related check point blockade mechanism can be a candidate for reverting cancer immunotherapy resistant tumor cell to cancer immunotherapy sensitive tumor cell.

In some embodiments, the compound can be administered to the patient substantially simultaneously with the cancer immunotherapy. In other embodiments, the compound can be administered to the patient at least 3 days, at least 7 days, at least 2 weeks, or even at least 1 month after the beginning of the cancer immunotherapy. In these embodiments, it is also contemplated that another dose/schedule of the cancer immunotherapy can be followed by the administration of the compound such that the cancer cells with reverted sensitivity to the cancer immunotherapy by the compound can be further and effectively treated by the cancer immunotherapy.

For example, patient A suffering from a non-small cell lung cancer is treated with the first stage of cancer immunotherapy using an oncolytic virus. Patient A's blood serum can be obtained 24 hours before, 24 hours after, and 3 days after the oncolytic virus treatment. Additionally, a blood serum from a healthy individual who is in the same age range of the patient A could be obtained for further comparison. From the patient A's blood serum, cell free RNA was purified and amplified by real time RT-PCR, using random primers (to amplify substantially all cell free RNA) or gene-specific primers (to amplify RNA of specific gene). Then amplified cell free RNA(s) are quantified and characterized. As an exemplary result, the expression level of subtype C HMGB1 mRNA (specific to lung) is increased for 30% compared to other subtypes of HMGB1 in 24 hours after the oncolytic virus treatment, and further increased for 50% in 3 days after the oncolytic virus treatment compared to the subtype C HMGB1 mRNA expression level of the patient before the oncolytic virus treatment. The similar increase could be found compared to the subtype C HMGB1 mRNA expression level of the healthy individual. In addition, no change in expression level of any other subtype of HMGB1 is observed.

The inventors contemplate that patient A may develop some resistance to the immune system activation (e.g., NK cell activation, T cell activation, etc.) within a period of time after the first stage of oncolytic virus treatment. For example, within a month, 3 months, or 6 months after the first stage of oncolytic virus treatment, decrease of the effectiveness of oncolytic virus treatment can be determined by clinical observation of increase of tumor size or by altered quantity and/or subtype of cell free RNA in a patient (e.g., increased expression of subtype C HMGB1 mRNA over two weeks and decreased below the pre-treatment level of HMGB1 mRNA expression, etc.). In this case, the patient A may be treated with compound X at a dose (10 mg per day, etc.) and schedule (e.g., once a day for 3 days, etc.) that is expected to be sufficient to change the immune-resistant cell to immune-susceptible cells. Then, the patient A can be treated for a second stage of oncolytic virus treatment. In some embodiments, the dose and schedule of the second stage of oncolytic virus treatment can be same or similar to the first stage of oncolytic virus treatment. Yet, it is also contemplated that the dose and schedule between the first and second oncolytic virus treatment may be different based on the prognosis of the tumor.

Patient A's blood serum can be obtained 24 hours before, 24 hours after, and 3 days after the second stage of oncolytic virus treatment. From the patient A's blood serum, cell free RNA was purified and amplified by real time RT-PCR, using random primers (to amplify substantially all cell free RNA) or gene-specific primers (to amplify RNA of specific gene). Then amplified cell free RNA(s) are quantified and characterized. As an exemplary result, the expression level of subtype C HMGB1 mRNA (specific to lung) is increased for 40% compared to other subtypes of HMGB1 in 24 hours after the second oncolytic virus treatment compared to 24 hours before the second oncolytic virus treatment, and further increased for 50% in 3 days after the second oncolytic virus treatment compared to 24 hours before the second oncolytic virus treatment. In addition, the expression level of subtype C HMGB1 mRNA in 3 days after the second oncolytic virus treatment is similar or less than 10% different compared to 3 days after the first oncolytic virus treatment. Such results can provide an indication that compound X was effective at least in increasing the effectiveness of the oncolytic virus treatment, potentially by reverting cancer immunotherapy resistant tumor cell to cancer immunotherapy sensitive tumor cell. Further those results can be associated with the clinical observation confirming that oncolytic virus treatment combined with compound X is effective (more effective than oncolytic virus treatment alone) by determining the reduced tumor size or reduced metastasis of the tumor cells in a specific location in the patient.

Consequently, the inventors further contemplate that, based on the increased or decreased cell free RNA expression level that are associated with the effectiveness of the treatment regimen and/or treatment (e.g., immune therapy, checkpoint inhibitor, chemotherapy, recombinant neoepitope vaccine, an oncolytic virus, etc.), one or more further treatment regimen and/or a treatment for the next round of the treatment plan can be determined and administered to the patient. For example, if patient A showed increased expression of subtype C HMGB1 mRNA upon oncolytic virus treatment with compound X, the treatment regimen for the next round of treatment plan can be determined to include compound X with oncolytic virus treatment or other types of immune therapy (e.g., recombinant neoepitope vaccine, etc.). In such scenario, the patient can be administered with such treatment regimen in a dose and schedule to sufficient to further increase or maintain the post-treatment level of the expression level of HMGB1 mRNA.

It should be apparent to those skilled in the art that many more modifications besides those already described are possible without departing from the inventive concepts herein. The inventive subject matter, therefore, is not to be restricted except in the scope of the appended claims. Moreover, in interpreting both the specification and the claims, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced. As used in the description herein and throughout the claims that follow, the meaning of “a,” “an,” and “the” includes plural reference unless the context clearly dictates otherwise. Also, as used in the description herein, the meaning of “in” includes “in” and “on” unless the context clearly dictates otherwise. Where the specification claims refers to at least one of something selected from the group consisting of A, B, C . . . and N, the text should be interpreted as requiring only one element from the group, not A plus N, or B plus N, etc.

What is claimed is:

1. A method for determining prognosis of a cancer immunotherapy, comprising:
obtaining a bodily fluid of a patient treated with the cancer immunotherapy;
identifying an expression level of a cell free RNA of at least one cancer related gene in the bodily fluid of the patient; and
associating the expression level of the cell free RNA with the prognosis of the cancer immunotherapy,
wherein the cell free RNA is mRNA encoding a protein selected from a group consisting of HMGB1, MUC1, VWF, MMP, CRP, PBEF1 TNF-α, TGF-β, PDGFA, and hTERT.

2. The method of claim 1, wherein the bodily fluid is selected from a group consisting of blood, serum, plasma, mucus, cerebrospinal fluid, and urine.

3. The method of claim 1, wherein the cancer immunotherapy is a treatment of the individual with at least one of a recombinant neoepitope vaccine, an oncolytic virus, or a checkpoint inhibitor.

4. The method of claim 1, wherein the cell free RNA is at least one of ctRNA or cfRNA.

5. The method of claim 4, wherein the cell free RNA is mRNA encoding an inflammation-related protein.

6. (canceled)

7. The method of claim 1, wherein the mRNA encoding HMGB1 comprises a plurality of alternative splicing variants.

8-13. (canceled)

14. The method of claim 1, wherein the identifying the quantity includes amplifying a signal of cell free RNA by real time, quantitative RT-PCR.

15-18. (canceled)

19. A method for identifying a location of a tumor that is susceptible to a cancer immunotherapy, comprising:
obtaining a bodily fluid of a patient treated with the cancer immunotherapy;
identifying an expression level and a subtype of a cell free RNA of at least one cancer related gene in the bodily fluid of the patient; and
associating the expression level and the subtype of the cell free RNA with the location of a tumor;
wherein the cell free RNA is mRNA encoding a protein selected from a group consisting of HMGB1, MUC1, VWF, MMP, CRP, PBEF1 TNF-α, TGF-β, PDGFA, and hTERT.

20. The method of claim 19, wherein the bodily fluid is selected from a group consisting of blood, serum, plasma, mucus, cerebrospinal fluid, and urine.

21. The method of claim 19, wherein the cancer immunotherapy is a treatment of the individual with at least one of a recombinant neoepitope vaccine, an oncolytic virus, or a checkpoint inhibitor.

22. The method of claim 21, wherein the cell free RNA is at least one of ctRNA or cfRNA.

23. The method of claim 22, wherein the cell free RNA is mRNA encoding an inflammation-related protein.

24-50. (canceled)

51. A method for identifying a compound effective to revert immune therapy resistant tumor cell to immune therapy sensitive tumor cell, comprising:
obtaining a bodily fluid of a patient treated with the cancer immunotherapy and the compound;
identifying an expression level of a cell free RNA of at least one cancer related gene in the bodily fluid of the patient; and
associating the expression level of the cell free RNA with the effectiveness of the compound in reverting immune therapy resistant tumor cell to immune therapy sensitive tumor cell;
wherein the cell free RNA is mRNA encoding a protein selected from a group consisting of HMGB1, MUC1, VWF, MMP, CRP, PBEF1 TNF-α, TGF-β, PDGFA, and hTERT.

52. The method of claim 51, wherein the bodily fluid is selected from a group consisting of blood, serum, plasma, mucus, cerebrospinal fluid, and urine.

53. The method of claim 51, wherein the cancer immunotherapy is a treatment of the individual with at least one of a recombinant neoepitope vaccine, an oncolytic virus, or a checkpoint inhibitor.

54. The method of claim 51, wherein the cancer immunotherapy is a treatment of the individual with an oncolytic virus.

55. The method of claim 51, wherein the cancer immunotherapy is a treatment of the individual with a checkpoint inhibitor.

56-73. (canceled)
